(19) United States
(12) Patent Application Publication
(10) Pub. No.: US 2019/0374367 A1
(43) Pub. Date: Dec. 12, 2019

(54) LUMBAR SUPPORT BELT

(71) Applicant: GIBAUD, Saint Etienne (FR)

(72) Inventors: Laurent DEVILLERS, Brindas (FR); Guillaume DUPORT, Saint Etienne (FR)

(21) Appl. No.: 16/434,875

(22) Filed: Jun. 7, 2019

(30) Foreign Application Priority Data
Jun. 11, 2018 (FR) 18/55053

(51) Int. Cl. A61F 5/02

(52) U.S. Cl. CPC A61F 5/028

(57) ABSTRACT

A lumbar belt having two branches, wherein each branch includes two elastic strips which are superimposed according to the anterior-posterior direction and which extend from the lateral end portion to the medial end portion of the branch. A first strip is upwardly inclined at an angle α with respect to the longitudinal axis of the belt, and a second strip is downwardly inclined at an angle θ with respect to the longitudinal axis of the belt. The lower lateral corner of the second strip is offset vertically upwards with respect to the lower lateral corner of the first strip, and the lower edge of the branch has a lower indentation configured to be located above a thigh of the wearer.

### BACKGROUND

The present invention concerns a lumbar support belt.

Such a belt is intended for people suffering from a low back pain, in particular. When it is properly positioned, the belt allows, on the one hand, reducing the pain felt by the user and, on the other hand, holding the user in a proper position, in particular thanks to the support of his lumbar region and his abdominal region.

FIG. 1 illustrates a lumbar support belt 100 of the prior art. The belt 100 includes two branches 101, 102 intended to be positioned on either side of the lower portion of the trunk of a wearer. The branches 101, 102 are linked at their medial end portion 103, whereas their lateral end portions 104 are provided with complementary attaching means 105, 105′ to enable them to be assembled when they are placed on each other in the abdominal area of the wearer.

Conventionally, the belt 100 has a median portion higher than the rest of the belt, so as to form a satisfactory bearing against the back of the wearer. Furthermore, the lower edge 107 of each branch 101, 102 has an indentation 108 which, when the belt 100 is worn, is located above a thigh of the wearer. Such an indentation 108 enables the passage of thighs in the seated position of the wearer, thus avoiding the belt bending on the thigh and/or moving up along the trunk of the wearer. The belt 100 may have an upper edge 109 symmetrical to the lower edge 108 with respect to a longitudinal axis 110 of the belt 100, the upper edge 109 thus also having an indentation 111 on each branch 101, 102.

This aforementioned shape of the belt 100 can be obtained from a single textile piece, as illustrated in FIG. 1. Such an embodiment however requires significant and therefore expensive making operations, in particular steps of cutting to the appropriate shape, of sewing and of edge working.

### SUMMARY

The present invention aims at overcoming the above-mentioned drawbacks. In particular, an object of the invention is to provide a belt having the specific shape required for the wearing comfort and the effectiveness of holding, but which has a simplified and less expensive design.

To this end, the invention concerns a lumbar support belt which has a vertical median axis of symmetry and a longitudinal axis, and which includes two branches intended to be positioned on either side of the lower portion of the trunk of a wearer. Each branch has a medial end portion and a lateral end portion, the medial end portions being linked to each other in the median area of the belt and the lateral end portions being provided with complementary attaching means to enable them to be assembled when they are placed on each other in the abdominal area of the wearer. The belt, when it is flat and in the unstressed state, is such that each branch includes:

a first elastic strip which extends from the lateral end portion to the medial end portion of the branch, according to a general direction upwardly inclined at an angle α with respect to the longitudinal axis of the belt;
a second elastic strip which extends from the lateral end portion to the medial end portion of the branch, according to a general direction downwardly inclined at an angle β with respect to the longitudinal axis of the belt;
the first and second strips being superimposed, over a portion of their surface, according to the anterior-posterior direction;

In addition, the first and second strips of each branch are configured and arranged with the lower lateral corner of the second strip which is offset vertically upwards with respect to the lower lateral corner of the first strip, so that, when the belt is flat and in the unstressed state, the branch has a non-rectilinear lower edge successively formed, from the lateral end portion to the medial end portion by:

an upwardly inclined portion formed by a portion of the lower edge of the first strip;
then a downwardly inclined portion formed by a portion of the lower edge of the second strip;
the lower edge of the strip thus having a lower indentation configured to be located above a thigh of the wearer.

Thus, in the belt according to the invention, it is simply necessary to superimpose strips and assemble them according to a particular configuration conferring the desired specific geometry, in particular the presence of the lower indentations. No operation of cutting the strips or of edge working being necessary for forming the indentations, the making of the belt is simple and inexpensive, yet without degrading the wearing comfort or the effectiveness of support. Besides the financial gain, the fact of not having to cut the strips in order to form the indentations also guarantees the integrity of the textiles.

In practice, the angles α and β are non-zero and preferably smaller than 60°.

The term “corner” refers to the portion of the surface of the strip located in the immediate vicinity of the lateral edge and the lower edge; this corner does not necessarily form an angle area.

Moreover, it should be noted that the medial end portions, if they are linked to each other, are not necessarily directly linked (but possibly via an intermediate part) and/or are not necessarily securely linked (but with a possibility of relative adjustment for example).

It can also be provided that the first and second strips of each branch are configured and arranged with the upper lateral corner of the second strip which is offset vertically upwards with respect to the upper lateral corner of the first strip, so that, when the belt is flat and in the unstressed state, the branch has a non-rectilinear upper edge successively formed, from the lateral end portion to the medial end portion by:

a downwardly inclined portion formed by a portion of the upper edge of the second strip;
then an upwardly inclined portion formed by a portion of the upper edge of the first strip;
the upper edge of the branch thus having an upper indentation substantially facing the lower indentation along the vertical direction.

Such a configuration improves further the comfort in a sitting position by limiting the bearing on the ribs of the wearer.

According to a possible embodiment, each of the strips has an upper edge and a lower edge which are substantially rectilinear and parallel to each other. In other words, the strip has a constant height. In this case, the lower edge of the branch forms a broken line, the portions defining the indentation being segments.

The first and second strips of a branch can be substantially identical.

The angle α and the angle β are larger than 5°, preferably larger than 10°, and smaller than 30°, preferably smaller than 20°. For example, the angle α and/or the angle β may be close to 12 to 15°.

It can be provided that the angles α and β are substantially identical (in absolute value). If, in addition, the strips are identical, the longitudinal axis can then be an axis of symmetry of the belt.

According to a possible embodiment, the lower indentation is located in the central third of the corresponding branch, considered parallel to the longitudinal axis of the belt.

Furthermore, it can be provided that the lower indentation forms a downwardly open flattened V whose angle is comprised between 135 and 170°, preferably between 140 and 160°, or even between 150 and 160°.

The vertical offset between the lower and/or upper lateral corners of the strips may be comprised between 5 and 30% of the height of a strip or each of the strips, preferably between 10 and 25%, even more preferably between 15 and 20%.

For example, the first and second strips of a branch may have the same constant height.

According to a possible embodiment, the lateral edges of the first and second strips of a branch are assembled to each other so as to be partially superimposed along a line which is preferably substantially vertical.

In other words, these lateral edges coincide, at least over a portion of their length. The partial superimposition—in other words the relative offset of the lateral edges—allows creating the lower indentation. The lateral edges may be connected to an end tab which carries the attaching means on the other branch (itself possibly provided with an end tab).

Said line, instead of being strictly vertical, could be inclined with respect to the vertical, for example at an angle smaller than 20°.

Furthermore, the belt may include a back piece intended to be placed against the back of the wearer, the medial edges of the first and second strips of a branch being assembled to the back piece. They can be removably assembled to the back piece; thus, said medial edges are repositionable relative to the back piece, which allows changing the treatment height, or better targeting the compression area.

According to a possible embodiment, the medial edges of the first strips of the branches are assembled to each other in a first junction area, and the medial edges of the second strips of the branches are assembled to each other in a second junction area, said first and second junction areas being distinct and assembled to the back piece, preferably in a removable way.

The medial edges of the first and second strips of a branch may be assembled on the back piece, preferably along a line which for example substantially coincides with the vertical median axis of symmetry of the belt.

According to a symmetrical configuration, which may be a standard configuration but can be modifiable, the medial edges of said first and second strips of the branch are not superimposed but substantially adjacent. In other words, the lower medial corner of the first strip is located just above the upper medial corner of the second strip.

According to an embodiment, the medial edges of the first strip of the first branch and of the first strip of the second branch can be assembled to the back piece substantially along the same median vertical line. Similarly, the medial edges of the second strip of the first branch and of the second strip of the second branch can be assembled to the back piece substantially along the same median vertical line.

The first and second strips of a branch may be superimposed, in the anterior-posterior direction, so that said branch forms a continuous wall devoid of a vertical space between the first and second strips, at least over the portion of the branch separated from the back piece. In other words, at the back piece, there may be such a vertical space.

### DESCRIPTION OF DRAWINGS

Several possible embodiments of the invention are now described, as non-limiting examples, with reference to the appended figures:

FIG. 1 illustrates a lumbar support belt;

FIG. 2 is a posterior view of a lumbar support belt according to the invention, which is flat and in the unstressed state;

FIG. 3 is an anterior view of the belt of FIG. 2, which is flat and in the unstressed state;

FIGS. 4 and 5 show a person, respectively front and side views, wearing the belt of FIG. 2;

FIG. 6 is a view similar to FIG. 2, showing a variant of the belt.

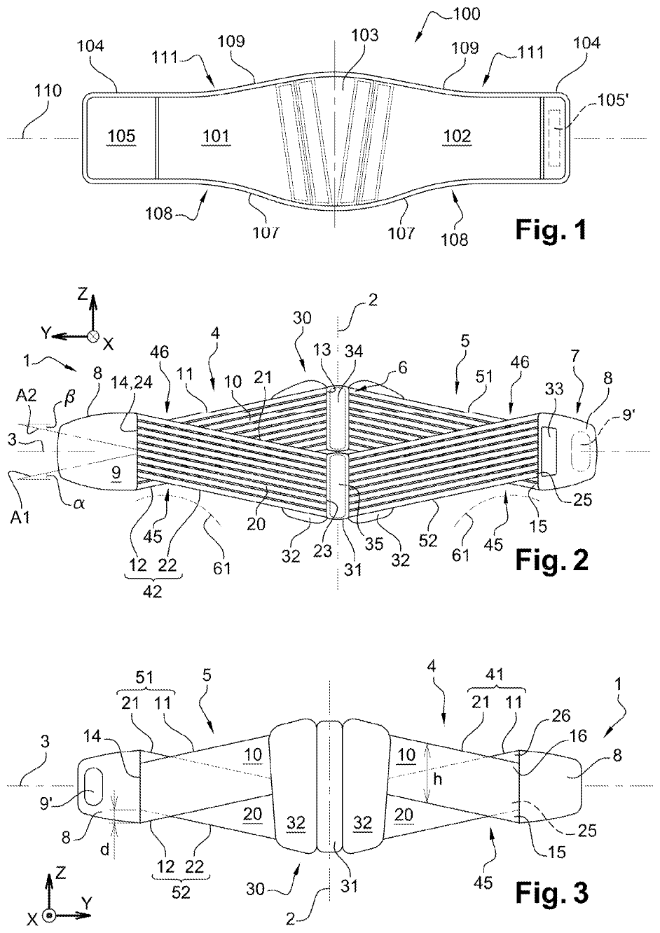

### DESCRIPTION OF VARIOUS EMBODIMENTS

FIG. 2 illustrates a lumbar support belt 1 according to the invention, when it is laid flat and in the unstressed state, that is to say in particular when no tension is exerted thereon.

The belt 1 has a vertical median axis of symmetry 2 and a longitudinal axis 3, which in some configurations of the belt 1 may be an axis of symmetry.

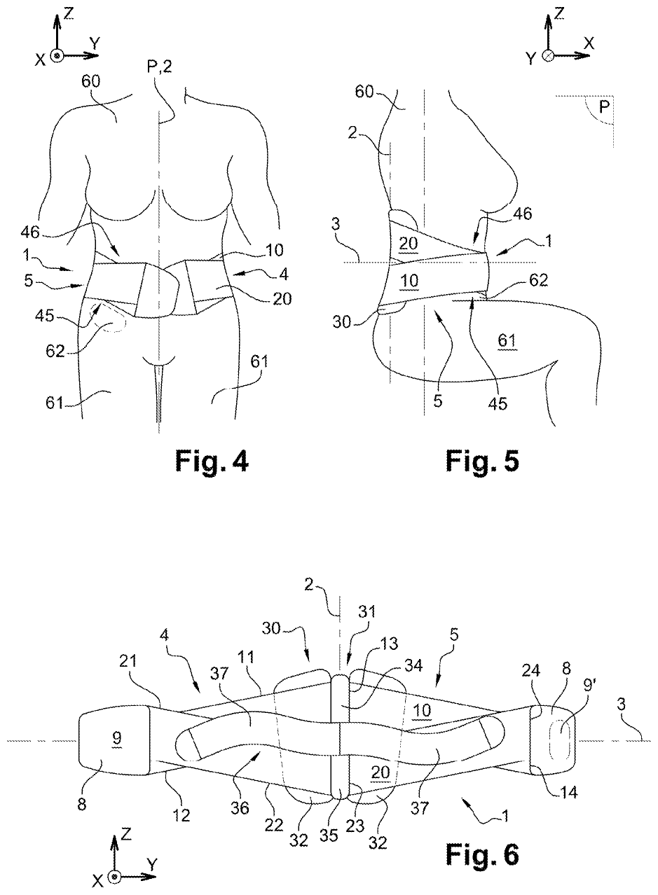

As illustrated in FIGS. 4 and 5, with reference to a wearer 60 of the belt 1, and in accordance with the anatomical reference system, the direction Z is defined as the vertical direction, the direction X as the anterior-posterior direction, and the direction Y as the horizontal direction orthogonal to X. The wearer 60 further has a midplane P parallel to (X, Z).

Thus, when the belt 1 is worn, the vertical median axis of symmetry 2 is substantially vertical and located in the plane P, and the longitudinal axis 3—then wound around the wearer 60—is substantially horizontal.

The terms “up”, “down”, “height”, “lower”, “upper” and the like are used with reference to the direction Z.

The terms “left”, “right”, “medial” and “lateral” are used with reference to the direction Y. An element will be referred to as “medial” if it is located closer to the vertical median axis of symmetry 2 or the plane P than another element, then referred to as “lateral”.

The terms “anterior”, “posterior” and the like are used with reference to the direction X.

The belt 1 comprises two branches intended to be positioned on either side of the lower portion of the trunk of a wearer, namely a left branch 4 and a right branch 5. Each branch 4, 5 has a medial end portion 6 and a lateral end portion 7.

The medial end portions 6 of the branches 4, 5 are linked to each other in the median area of the belt 1, that is to say the area located at the center of the belt 1 when it is laid flat, this area corresponding to the dorsal area of the wearer 60 in the use position.

The lateral end portions 7 of the branches 4, 5 are provided with complementary attaching means 9, 9′ to enable them to be assembled when they are placed on each other in the abdominal area of the wearer 60, as illustrated in FIG. 4.

According to a possible embodiment, each of the lateral end portions 7 of the branches 4, 5 includes an end tab 8 which carries said complementary attaching means. For example, the posterior face of an end tab 8 may include a loop area 9 and the anterior face of the other end tab 8 may include a hook area 9′, to form a Velcro® type hooking. The end tabs 8 may be expandable or not. At least one of the end tabs 8 may include a finger-slit 33 allowing facilitating the tension of the branches 4, 5 by the wearer 60 during the set-up of the belt 1.

The belt 1 may also comprise a back piece 30 intended to be placed against the back of the wearer 60, as shown in FIG. 5.

The back piece 30 may be formed by a central portion 31 extending along the vertical median axis of symmetry 2, intended to face the spinal column of the wearer 60, and two lateral portions 32 intended to be placed on both sides of the spinal column. The back piece 30 may be entirely rigid; alternatively, the two lateral portions 32 may be rigid whereas the central portion 31 may be flexible. The lateral portions 32 may project forwardly relative to the central portion 31, to form a vertical splint for receiving the spinal column. Furthermore, the back piece 30 may include anti-slide elements and/or stiffening elements, and/or means promoting the breathability (for example, thanks to the material constituting the back piece 30 and/or to appropriate orifices).

Each branch 4, 5 of the belt 1 includes two elastic strips which are superimposed, over a portion of their surface, according to the anterior-posterior direction X. Thus, when the belt 1 is flat and in the unstressed state, there are:

a first elastic strip 10 which extends from the lateral end portion 7 to the medial end portion 6 of the branch, according to a general direction A1 upwardly inclined at an angle α with respect to the longitudinal axis 3 of the belt 1;

a second elastic strip 20 which extends from the lateral end portion 7 to the medial end portion 6 of the branch, according to a general direction A2 downwardly inclined at an angle β with respect to the longitudinal axis 3 of the belt 1.

According to one embodiment, the strips 10, 20 are identical, and substantially rectangular. Each strip therefore has an upper edge and a lower edge which are substantially rectilinear and parallel to each other, and has a height h—that is to say the distance between the lower edge and the upper edge, generally according to the direction Z—substantially constant. More specifically:

the first strip 10 includes an upper edge 11, a lower edge 12, a medial edge 13 and a lateral edge 14;
the second strip 20 includes an upper edge 21, a lower edge 22, a medial edge 23 and a lateral edge 24.

Because of the inclination of the strips 10, 20 with respect to the longitudinal axis 3 of the belt 1, the medial edge and the lateral edge of a strip, which may be oriented vertically, may be not exactly orthogonal to the upper and lower edges.

The strips 10, 20 may be made of an elastic textile of an appropriate liveliness. They can be obtained by cutting to the desired length a long rectangular strip, transversely to said strip, which does not generate significant making work. However, no cutting is necessary at the lower or upper edges of the strips 10, 20 to obtain the desired shape, as explained hereinafter.

The strips 10, 20 of a given branch 4, 5 are assembled to each other by their lateral edges 14, 24, which may for example be sewn on the corresponding end tab 8, preferably along a substantially vertical line.

Furthermore, the medial edges 13, 23 of the first and second strips 10, 20 of each branch 4, 5 are assembled to the back piece 30.

According to a possible embodiment, as illustrated in FIG. 2, the medial edges 13 of the first strips 10 of the two branches 4, 5 are assembled to each other in a first junction area 34, and the medial edges 23 of the second strips 20 of the two branches 4, 5 are assembled to each other in a second junction area 35. Typically, the medial edges 13, 23 may be sewn on the corresponding junction area 34, 35. The junction areas 34, 35 are distinct and preferably removably assembled to the back piece 30. These junction areas 34, 35 may for example include, on their anterior face, removable attaching means on the back piece 30. The attachment can be done in the central portion 31 of the back piece 30, along a line which is for example substantially coincident with the vertical median axis of symmetry 2 of the belt 1.

Thus, concretely, it is possible to detach a junction area 34, 35 of the back piece 30, then assemble it again to the back piece 30 in a different position. This allows increasing or decreasing the angles α, β of inclination of the strips 10, 20, according to the needs of the wearer 60. The symmetry of the branches 4, 5 with respect to the vertical median axis of symmetry 2 of the belt 1 may however be preserved.

Alternatively, the medial edges 13, 23 of the strips 10, 20 could be sewn directly on the back piece 30, without possibility of adjustment of the assembly height.

In the embodiment represented in FIG. 2, the strips 10, 20 have the same constant height h, which is about half the height of the back piece 30. Nonetheless, this embodiment is not limiting.

Furthermore, in FIG. 2, the junction areas 34, 35 are assembled to the back piece 30 on each other, in an adjacent manner but without superimposition. Then, a belt 1 symmetrical with respect to the longitudinal axis 3 is obtained, the angles α and β being identical, and the end tabs 8 being centered vertically with respect to the back piece 30. This configuration can however be modified by displacement of the junction areas 34, 35 relative to the back piece 30.

According to a feature of the belt 1 according to the invention, the strips 10, 20 of each branch 4, 5 are assembled so that the lower lateral corner 25 of the second strip 20 is offset vertically upwards with respect to the lower lateral corner 15 of the first strip 10. Concretely, this can be obtained by sewing strips 10, 20 at different heights on the corresponding end tab 8.

Thus, when the belt 1 is flat and in the unstressed state, as shown in FIGS. 2 and 3, each branch 4, 5 has a non-rectilinear lower edge 42, 52 successively formed, from the lateral end portion 7 to the medial end portion 6, by:

an upwardly inclined portion formed by a portion of the lower edge 12 of the first strip 10;
then a downwardly inclined portion formed by a portion of the lower edge 22 of the second strip 20.

It follows that the lower edge 42, 52 of the branch 4, 5 has a lower indentation 45. Furthermore, the belt 1 is configured so that, when it is worn, each of the lower indentations 45 is located above a thigh 61 of the wearer 60, that is to say in the inguinal region 62 of the wearer 60, as illustrated in FIGS. 4 and 5.

Thanks to this, when the wearer 60 sits, the upper portion of his thighs 61 can fit in a lower indentation 45. Thus, on the one hand, the discomfort is considerably limited for the wearer in the sitting position. On the other hand, the risk that the belt 1 moves up is significantly reduced because the thighs 61 do not bear against the lower edge of the belt 1 and therefore do not tend to push it upwardly.

In FIG. 2, the position of the thighs 61 of the wearer 60 in the seated position is schematized by a dotted line.

In practice, the lower indentation 45 is preferably located in the central third of the corresponding branch 4, 5, considered parallel to the longitudinal axis 3 of the belt 1. In this way, once the belt 1 is set up on the wearer 60, the lower indentation 45 will be in the appropriate position above a thigh 61.

The vertical offset d between the lower lateral corner 25 of the second strip 20 and the lower lateral corner 15 of the first strip 10 (see FIG. 3) may be comprised between 5 and 30% of the height h of the strips 10, 20, preferably between 10 and 25%, even more preferably between 15 and 20%.

Moreover, the angle α and the angle β may be larger than 5°, preferably larger than 10°, and smaller than 30°, preferably smaller than 20°.

In the illustrated embodiment, the indentation 45 forms a broken line, to the extent that the lower edges 12, 22 of the first and second strips 10, 20 are rectilinear. Thus, the indentation 45 forms a downwardly open flattened V. As example, the angle formed by this V may be comprised between 135 and 170°, preferably between 140 and 160°, or even between 150 and 160°.

Moreover, it may be provided that the upper lateral corner 26 of the second strip 20 is offset vertically upwards with respect to the upper lateral corner 16 of the first strip 10. This is automatically the case if the first and second strips 10, 20 have the same height and that their lower lateral corners 15, 25 are offset as previously described. The offset d is then the same between the lower lateral corners 15, 25 and between the upper lateral corners 16, 26.

Thus, when the belt 1 is flat and in the unstressed state, each branch 4, 5 has a non-rectilinear upper edge 41, 51 successively formed, from the lateral end portion 7 to the medial end portion 6, by:

a downwardly inclined portion formed by a portion of the upper edge 21 of the second strip 20;
then an upwardly inclined portion formed by a portion of the upper edge 11 of the first strip 10.

Consequently, the upper edge 41, 51 of the branch 4, 5 has an upper indentation 46. The latter substantially faces the lower indentation 45 along the vertical direction Z. The relative position of the indentations 45, 46 can slightly vary according to the angles α and β, in particular because of the height adjustment of the position of the junction areas 34, 35 on the back piece 30.

As shown in FIGS. 4 and 5, the upper indentation 46 allows further improving the wearing comfort, particularly in a sitting position, by limiting the bearing on the ribs of the wearer 60.

Regardless of the assembly position of the junction areas 34, 35 on the back piece 30, it is preferable to provide that the first and second strips 10, 20 of each of the branches 4, 5 are superimposed, according to the anterior-posterior direction X, so that each of the branches 4, 5 forms a continuous wall devoid of a vertical space between the first and second strips 10, 20, at least over the portion of the branch 4, 5 separated from the back piece 30. This meets requirements in terms of comfort for the wearer 60 and effectiveness of the support.

FIG. 6 represents a variant of the belt 1, which includes in addition an additional tightening device 36, for example in the form of two lateral straps 37. Thus, the branches 4, 5 can be tightened without excess and, when this is necessary, it is possible to temporarily increase the tightening intensity, typically when the wearer 60 will make a significant effort, carry a heavy load, etc., by pulling the two lateral straps 37 and by fastening them each on the corresponding branch 4, 5 by appropriate hooking means. Subsequently, it is possible to return to the initial tightening, of a lower intensity, by detaching the additional tightening device 36.

Thus, the invention provides a crucial improvement to the state of the art, by providing a lumbar support belt of a simple and inexpensive design yet allowing obtaining the required comfort and effectiveness. More specifically, the traditionally present lower indentation is simply obtained by a specific relative arrangement of the superimposed strips forming each of the branches of the belt. No cutting of the strips at its lower or upper edges is necessary, which guarantees their integrity and considerably simplifies the making thereof.

It goes without saying that the invention is not limited to the embodiments described hereinabove as examples but that it comprises all technical equivalents and variants of the means described as well as the combinations thereof.

1. A lumbar support belt having a vertical median axis of symmetry and a longitudinal axis, the belt comprising:
two branches arranged to be positioned on either side of the lower portion of the trunk of a wearer, the branches each having a medial end portion and a lateral end portion, the medial end portions being linked to each other in the median area of the belt and the lateral end portions being provided with complementary attaching means to enable them to be assembled when they are placed on each other in the abdominal area of the wearer, the belt, when it is flat and in the unstressed state, being such that each branch includes:
a first elastic strip extending from the lateral end portion to the medial end portion of the branch, according to a general direction upwardly inclined at an angle α with respect to the longitudinal axis of the belt;
a second elastic strip extending from the lateral end portion to the medial end portion of the branch, according to a general direction downwardly inclined at an angle β with respect to the longitudinal axis of the belt;
the first and second strips being superimposed, over a portion of their surface, in the anterior-posterior direction;
wherein the first and second strips of each branch are configured and arranged with the lower lateral corner of the second strip which is offset vertically upwards with respect to the lower lateral corner of the first strip, so that, when the belt is flat and in the unstressed state, the branch has a non-rectilinear lower edge successively formed, from the lateral end portion to the medial end portion by:
an upwardly inclined portion formed by a portion of the lower edge of the first strip;
then a downwardly inclined portion formed by a portion of the lower edge of the second strip;
the lower edge of the strip thus having a lower indentation configured to be located above a thigh of the wearer.

2. The belt according to claim 1, wherein the first and second strips of each branch are configured and arranged with the upper lateral corner of the second strip which is offset vertically upwards with respect to the upper lateral corner of the first strip, so that when the belt is flat and in the unstressed state, the branch has a non-rectilinear upper edge successively formed, from the lateral end portion to the medial end portion by:
a downwardly inclined portion formed by a portion of the upper edge of the second strip;
then an upwardly inclined portion formed by a portion of the upper edge of the first strip;
the upper edge of the branch thus having an upper indentation substantially facing the lower indentation along the vertical direction.

3. The belt according to claim 1, wherein each of the strips has an upper edge and a lower edge which are substantially rectilinear and parallel to each other.

4. The belt according to claim 1, wherein the first and second strips of a branch are substantially identical.

5. The belt according to claim 1, wherein the angle α and the angle β are larger than 5°.

6. The belt according to claim 1, wherein the angles α and β are substantially identical.

7. The belt according to claim 1, wherein the lower indentation is located in the central third of the corresponding branch, considered parallel to the longitudinal axis of the belt.

8. The belt according to claim 1, wherein the lower indentation forms a downwardly open flattened V whose angle is comprised between 135 and 170°.

9. The belt according to claim 1, wherein the vertical offset between the lower and/or upper lateral corners of the strips is comprised between 5 and 30% of the height of a strip or each of the strips.

10. The belt according to claim 1, wherein the lateral edges of the first and second strips of a branch are assembled to each other so as to be partially superimposed along a line which is substantially vertical.

11. The belt according to claim 1, wherein it includes a back piece intended to be placed against the back of the wearer, and in that the medial edges of the first and second strips of a branch are assembled to the back piece.

12. The belt according to claim 11, wherein the medial edges of the first and second strips of a branch are removably assembled on the back piece.

13. The belt according to claim 11, wherein the medial edges of the first strips of the branches are assembled to each other in a first junction area, and in that the medial edges of the second strips of the branches are assembled to each other in a second junction area, said first and second junction areas being distinct and assembled to the back piece, removably assembled to the back piece.

14. The belt according to claim 11, wherein the medial edges of the first and second strips of a branch are assembled on the back piece, along a line which is for example substantially coincident with the vertical median axis of symmetry of the belt.

15. The belt according to claim 11, wherein the first and second strips of a branch are superimposed, according to the anterior-posterior direction, so that said branch forms a continuous wall devoid of a vertical space between the first and second strips, at least over the portion of the branch separated from the back piece.
